(19) United States
(12) Patent Application Publication
(10) Pub. No.: US 2025/0195256 A1
(43) Pub. Date: Jun. 19, 2025

(54) PANTS THAT CAN ACCOMMODATE A CATHETER

(71) Applicant: Nitza Agam, Daly City, CA (US)

(72) Inventor: Nitza Agam, Daly City, CA (US)

(21) Appl. No.: 18/978,458

(22) Filed: Dec. 12, 2024

Related U.S. Application Data
(63) Continuation-in-part of application No. 18/143,199, filed on May 4, 2023.

(51) Int. Cl. A61F 5/44; A41D 1/06

(52) U.S. Cl. CPC A61F 5/4408; A41D 1/06; A41D 2300/30; A41D 2400/48

(57) ABSTRACT

Pants that can accommodate a catheter including a panel that is a covering component of a pant leg that can provide access to the at least one collection bag on a frontal anterior thigh of the wearer, wherein the panel provides partial and full access to the at least one collection bag, wherein the panel has a lateral edge that is coextensive with a lateral seam of a pant, a partial perimeter outer edge that is closed by a continuous zipper tape with a stop, where the partial perimeter outer edge of the panel is substantially coextensive with an inner edge of the pant leg, the continuous zipper tape has a zipper slide assembly with a zipper slide having a locking pull configured to open and close the panel and continuous zipper tape.

This Application is a Continuation-in-Part (CIP) of application Ser. No. 18/143,199, filed May 4, 2023.

### TECHNICAL FIELD

The present invention in general is pants for an individual who has been fitted with a catheter that drains urine from their bladder into at least one collection bag that is mounted on a frontal region of one or more of their legs. The catheter is typically a Foley catheter. The invented pants facilitate operation and care of the at least one collection bag.

### BACKGROUND OF THE INVENTION

Silverts sells Men's Easy Zipper Pants online at https://www.silverts.com/discover-silverts, stating that the pants are great for arthritis, catheters & paralysis. The pants have a waist with a drawstring, 1 back pocket & 2 side pockets, and functional fly zippers to just below the knee with a VELCRO closure from the knee down. The pant leg is open at the bottom for hemming the pant leg. Silvert advertises that his pants are versatile, great for knee surgery, hip replacement, broken legs & catheters. The pants have two-way zippers that open fully for easy access to casts and catheters and are easy for anyone in a reclining position. Silvert's Easy Touch Closures helps daily tasks such as dressing and undressing, and assisted dressing or independent dressing. Side zippers are secured at the waist with easy touch tabs and end above the ankle to allow for hemming. Also available are Open back pants for people who need help in getting dressed or have limited mobility. These open-back pants are traditional in fabrication, but with two overlapping panels on the backside that are closed using snaps in the waistband. With multiple snaps along the waistband they can be easily adjusted. There is no teaching for accessing a urine collection bag.

Buck-and-Buck sell a drainage bag cover, made by Adaptable Designs. The cover for a standard urinary drainage bag helps to maintain some privacy and dignity for the person who needs to carry the drainage bag around with them. The cover conceals the urine drainage from view, yet the drainage bag can be easily accessed for monitoring and emptying. Buck-and-Buck state that the need for a urinary catheter does not have to cause difficulty with wearing certain styles of clothing. Any of our pants for men and women can have a Velcro opening sewn into the inseam or side seam, whichever you prefer to make you more comfortable and to allow access for care and emptying of the urine collection bag. In order to maintain best infection control practice by not disconnecting the catheter from the drainage tubing since the catheter port is a potential path for entry of bacteria into the body, Buck-and-Buck teach a comfortable 12 inch hook and loop closure on the inseam of the slacks that allows the empty drainage bag to be passed through the leg opening. In this embodiment urine would exit through the inseam of the slacks, or the drainage bag is mounted on the slack's leg on an outside surface of the slacks. Alternatively, the drainage bag can be attached to the wheelchair. The integrity of the closed catheter and urinary drainage tubing can be maintained. A cover provides some privacy for the person wearing a urinary catheter.

### SUMMARY OF THE INVENTION

The invention is pants for an individual who has been fitted with a catheter, wherein the catheter drains urine from their bladder into at least one collection bag, is mounted on a frontal region of one or more of their legs. The catheter is typically a Foley catheter. The invented pants facilitate operation and care of the at least one collection bag.

An aspect of the invention is that it provides easy access to a lower portion of a collection bag, wherein a valve on an outlet can be controlled, therein enabling drainage of the collection bag.

A second aspect of the invention is that a length of flexible plastic tubing can be fitted onto a tubular extension of a collection bag's outlet, therein enabling discharge of urine to a commode, a bedpan, or any suitable vessel. A length of the flexible plastic tubing can be appropriately selected.

A third aspect of the invention is that it provides easy access to an upper portion of a collection bag, wherein an inlet to the collection bag is visibly connected to the catheter can be confirmed, and if there is or isn't any leakage. If there is leakage, the connection can be tightened until controlled, and a surrounding can be cleaned. A determination of whether a new collection bag may be needed. The user's skin can be cleaned.

A fourth aspect of the invention is that it provides easy access to a frontal view of the installed collection bag. If straps need to be adjusted, adjustments can be made, depending on how the collection bag is fastened, and potentially a new collection bag can be installed.

A fifth aspect of the invention is that the frontal view access to the collection bag can include a view of the straps that secure the collection bag to a leg, and the view can alternatively be limited to a partial portion on a single thigh or to a different partial portion both thighs.

A sixth aspect of the invention is that the pants are large enough to accommodate the wearer's the catheter, and made of a material that is sufficiently lightweight that the wearer can feel the catheter through the material, and shift the catheter as needed; and

A final aspect of the invention is that a selected zipper tape conforms to curved applications, therein providing panels under load will not yield, and less more resistant than Velcro to become fouled with hair or detritus.

### BRIEF DESCRIPTION OF THE DRAWINGS

The foregoing invention will become readily apparent by referring to the following detailed description and the appended drawing in which:

FIG. 1 is a frontal view of the invented pants, wherein at least one pant leg has a panel that provides access to a collection bag mounted frontally on a wearer's anterior thigh;

FIG. 2 is a frontal view of the invented pants that have a left panel and a right panel, wherein the illustrated panels are not limited, such that either or both panels can be closed as shown in FIG. 1, or partially open, or fully open; and for illustrative purposes one panel has an upper portion that is open, and the other panel has a lower portion of the other panel that is open;

FIG. 3 is a frontal view of a length of flexible plastic tubing fitted onto a tubular extension of the collection bag's drainage port outlet having a tubular extension, wherein the length of flexible plastic tubing is several inches long;

FIG. 4 is a frontal view of the invented pants, wherein currently the left panel is fully open therein enabling a full view of the collection bag, the drainage outlet, and the inlet (per se the collection bag is not an invented element of the instant invention, and therefore is shown with dashed lines); and as illustrated the right panel is partially open; and

FIG. 5 illustrates pants having a single zipper per pant leg in accordance with embodiments described herein;

FIG. 6 illustrates another embodiment of pants to accommodate a catheter as described herein; and

FIG. 7 illustrates yet another embodiment of pants to accommodate a catheter as described herein.

### DETAILED DESCRIPTION OF THE INVENTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Although the various exemplary embodiments have been described in detail with particular reference to certain exemplary aspects thereof, it should be understood that the invention is capable of other example embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

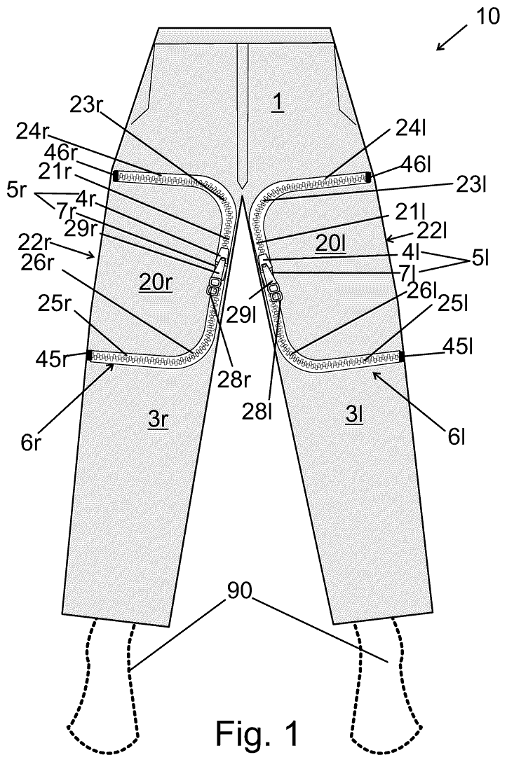

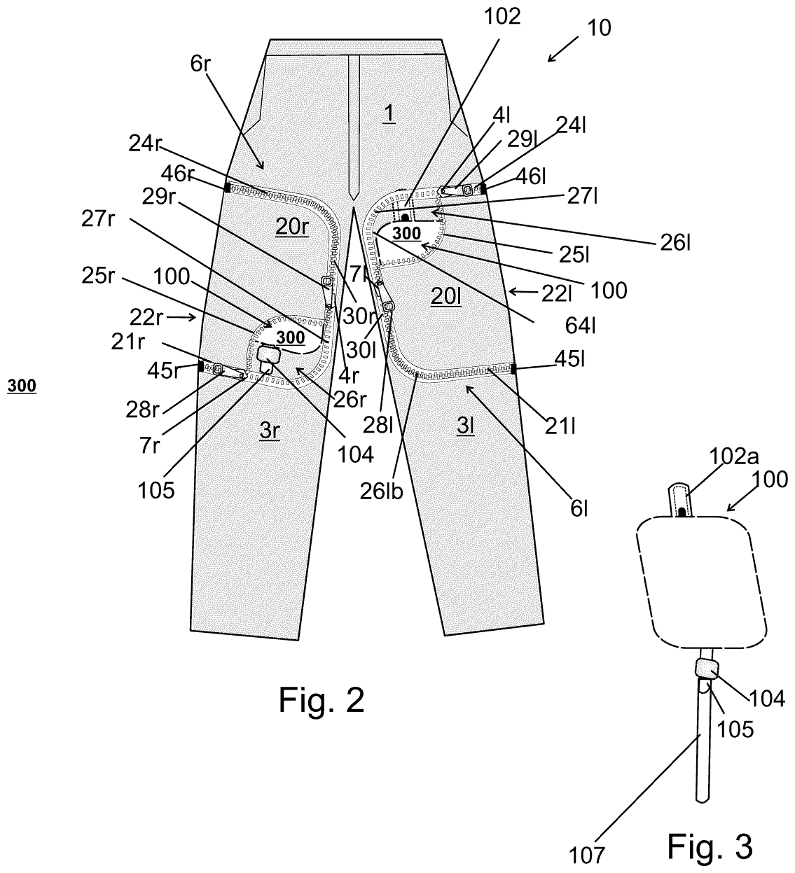

FIG. 1 is a frontal view of an embodiment 10 of the invented pants 1, wherein at least one pant leg (3r, 3l) has a panel 20r, 20l that provides access to a collection bag (aka urinary leg bag) with an inlet 102 connection to a catheter (not shown), and an outlet 104 with a valve that is used to drain the collection bag 100, as shown in FIGS. 2 and 3). Per se the collection bag is not an invented element if the invention, and therefore is shown with dashed lines to indicate this. Similarly, legs 90 are shown with dashed lines.

The illustrated pants 1 have a left panel 20l and a right panel 20r, therein potentially enabling use and access to two collection bags. Each panel 20r,20l is secured by a shared continuous zipper tape 6r,6l and a lateral seam 22r,22l of the legs 3r,3i of the pants 1. The shared continuous zipper tape 6r,6l include a medial section 21r, 21l that upwardly transition into upper curved section 23r, 23l; which laterally transitions into an upper horizontal section 24r, 24l ending in an upper stop 46r,46l. The medial section 21r, 21l also downwardly transitions into a lower curved sections 26r,26l; which laterally transitions into to a lower horizontal section 25r,25l ending in a lower stop 45r,45l. The shared continuous zipper tape spans an outer partial edge of a panel with an adjoining inner partial edge of an opening of the pant, therein connecting to the partial edge of the panel to the inner partial edge of the opening of the pant.

In this configuration, each pant leg has a continuous zipper tape, which taken with the lateral seams 22r,22l defines the panels 20r,20l. Each of the zipper tapes has a pair of zipper slide assemblies 5r,5l; where each zipper slide assembly includes a pair of opposing zipper slides 7r,4r and 7l,4l with pulls 28r,29r and 28l,29l for opening and closing all or partial portions of the zipper tapes 6r,6l. Note, each pair of opposing zipper slides 7r,4r and 7l,4l are aligned such that a pair of zipper slides are opposing and nose to nose. In this alignment when a left pair of slides are pulled apart, the left zipper tape 6l unzips and opens providing a portlet 300, and when the left pair of slides 7l,4l are pulled back together, the left zipper tape 6l closes, wherein opposing teeth on 6l are alternately interlocked. Likewise, when a right pair of slides 7r,4r are pulled apart, the right zipper tape 6r unzips and opens, also providing a portlet 300, and when the right pair of slides 7r,4r are pulled back together, the right zipper tape 6r closes, wherein opposing teeth on 6r are alternately interlocked.

The zipper assemblies 5r,5l are independent, and movement of the right slides 7r,4r does not affect movement of the left slides 7l,4l. This especially the case when the pulls 28r,29r and 28l,29l are locking pulls.

The zipper size is selected to be appropriate for the size of the pants. Smaller pants would typically have a size #4 or #4.5, which has a closed width of ˜4 mm. Size #5 is ˜5 mm, and is typically strong enough for most adults. A size #10 is generally only required for larger projects.

In the instant invention, the environment shouldn't be very harsh, so molded tooth zippers made from plastic, such as DuPont Delrin® Acetal are suitable resins to mold into a zipper tape. Exemplary manufactures include YKK® molded tooth zippers are called Vislon®; Lenzip® doesn't use a special term. The construction makes them strong and practically weatherproof, and urine proof. Molded tooth zippers are corrosion resistant.

Coil zippers utilize nylon teeth (Nylon® which originally is also a DuPont plastic), wherein the teeth are sewn onto the side tape with polyester thread. Coil zipper teeth are stronger and more flexible than molded tooth zippers, making them more easily formed into curved applications. Polyester thread will break down in the sun, so coil zippers should be kept covered to protect them from UV exposure in outdoor projects. Typical coil zippers are sizes #4.5, #5 and #10. Coil zipper tapes are advantageously nearly flat on the rear side.

Metal zippers have polished metal teeth that secure together on the zipper chain. Because they can corrode, they are only used indoors. Typically, they are used for bags and purses, backpacks, interior cushions and upholstery, and more. A potential downside of metal zippers is that metal zippers can have sharper edges, which potentially could scratch a collection bag, which is typically constructed of vinyl. Metal zippers are harder to stich as they will break a needle.

While coil zippers are preferred, they are not limiting as metal and molded tooth zippers can also be used in the disclosed invention. It is further anticipated that the opposing zipper tapes 6r,6l can include an overlying fabric tape and an underlying fabric tape onto the zipper tapes 6r,6l. Typically, basting tape is used to fasten zipper tapes to cloth materials, and fabric tapes. Basting tape is a narrow double-sided pressure sensitive adhesive tape on a release liner, often employed to facilitate mechanized stitching.

An example of a collection bag is the AMSure® Urinary Leg Bag. Its features include that the urine leg bag safely secures to the leg with two adjustable elastic leg straps. The catheter leg bag easily connects to any Foley catheter. It has leak proof seams, which ensures fluid containment. It has two flexible, high quality straps, to ensure secure attachment to either of the wearer's leg. The leg straps are soft and breathable. The urinary leg bag is an anti-reflux leg bag catheter bag, which helps improve patient safety by reducing urine backflow. It has either a push-pull or a twist-turn drainage port. The medium size has a potential volume of 600 ml. The large size has a potential volume of 900 ml. The push-pull drainage port (also called the T-Tap drainage port includes a tubular extension with a sloped opening 105.

It is anticipated that the instant invention 1 includes a length of flexible plastic tubing 107 fitted onto the tubular extension 105 collection bag's drainage port outlet 104 with a tubular extension 105 with. The length of flexible plastic tubing is several inches long, as illustrated in FIG. 3, therein extending the outlet below a wearer's knee, thereby facilitating periodic drainage directly into a commode or a bedpan.

FIG. 2 illustrates how an upper portion of the left panel 20l is opened. The zipper slide 4l is pulled, from an initial left central position of the left medial section 21l shown in FIG. 1, upward and through the upper left curve 27l, and then laterally to the upper horizontal section 24l, stopping at a point 24l, which is proximate to the stop 46l, which is proximate to a left seam 22l of the invented pants 1. The unzipped portion of the left panel 20l therein exposes an upper portion of the collection bag 100 and the inlet 102. The unzipped portion of panel 20l in FIG. 2 is bounded by a left set of inner teeth 25l and a left set of outer teeth 27l on the zipper tape 6l.

The unzipped portion of the zipper tape 6l provides an panel portlet 26l to the inlet 102 and to an upper portion of the collect bag. In the illustrated embodiment the outer teeth 27l are on an underlying fabric tape 641, which provides a protective buffer. Optionally, the inner teeth can also be recessed on an overlying fabric tape 651.

A preferred zipper type is a coil zipper, as the coil is relatively fine-toothed, and it provides a smooth zip. Coil zippers are stronger and more flexible than molded tooth zippers, making them suitably operative for the curved surface of the invention. The zipper size is selected based on the size of the invented pants. For most applications a size #5 is suitable.

Optionally, a protective fabric tape can be employed to keep the zipper covered, wherein the fabric tape protects the collection bag from contact with the zipper. Testing supports that Lenzip® sliders work with Lenzip® zipper chain and YKK® sliders work with a YKK® zipper chain.

FIG. 2 also illustrates a first operating characteristic of the opposing zipper slides, which is that as the opposing zipper slides are partially pulled apart, the continuous zipper tape unzips therein providing a partial opening, which is a panel portlet. The panel portlet provides partial access to the collection bag, wherein said partial perimeter outer edge of the panel separates from said inner edge of the pant leg; wherein the panel portlet can be closed by pulling the opposing zipper slides together

Another illustration of the panel portlet is in a lower portion of the right panel 20r. The zipper slide 7r has been pulled, from an initial right central position of the right medial section 21r shown in FIG. 1, downward and through the lower right curve 26r, and then laterally to the lower horizontal section 25r, stopping at a lower point 21rl, which is proximate to a right seam 22r of the invented pants 1. The unzipped portion of the right panel 20r therein exposes, via the panel portlet, a lower portion of the collection bag 100 and a drainage outlet 104 having a tubular extension 105. The unzipped portion of panel 20l in FIG. 2 is bounded by a left set of inner teeth 25l and a left set of outer teeth 27l, which define an access port 26l to the inlet 102 and to an upper portion of the collect bag.

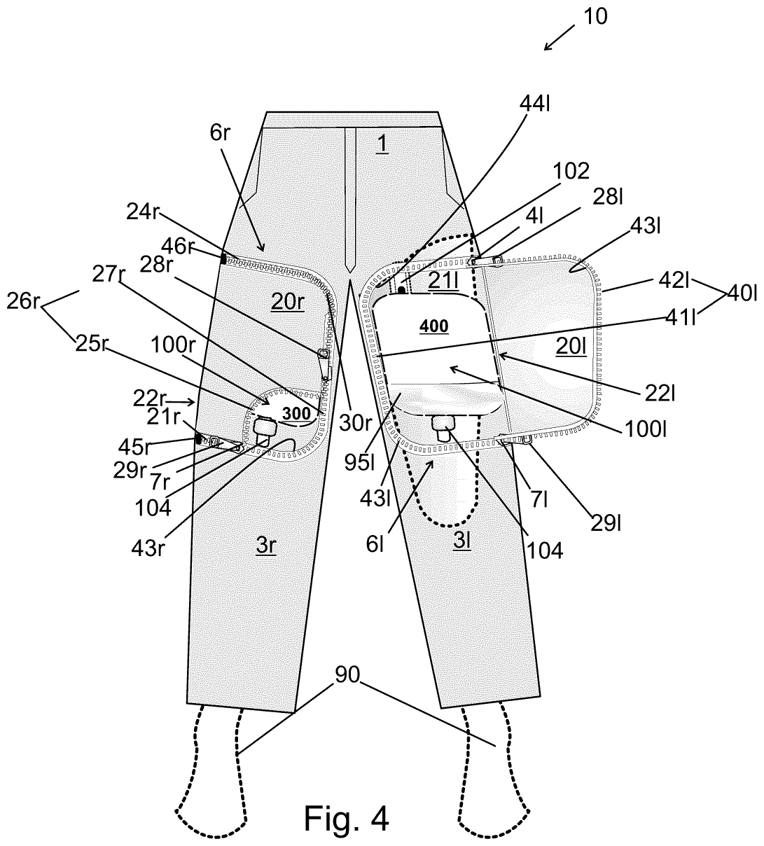

The invented pants 1 in FIG. 4 illustrates a second operating characteristic of the opposing zipper slides is when the opposing zipper slides are pulled apart until each abut an upper stop and a lower stop, the continuous zipper tape is unzipped such that there is a full opening, providing a port 400 to the entire collection bag.

The unzipped panel is folded laterally along the lateral seam of the pant leg, providing a port with full access to the collection bag via the port 400; wherein the left panel 20l is fully opened along the seam 22l, exposing an interior side 21l of pant 3i, when the left slides 4l,7l are pulled to the stops, see 45l,46l shown in FIG. 1 and FIG. 2. With the left panel fully opened one can view the collection bag 100, the inlet 102 and the outlet 104. A view of how much urine has been collected, a partial view of the straps is possible, and an inspection of the connection of the inlet to a catheter. The wearer's legs 90 are shown. The collection bag 100 shown in the left panel has a volume 95l of urine. The collection bag 100 shown in the right panel has no urine. Unless the right collection bag 100r is new, no urine can be indicative that there is a problem, like a leak at the inlet that connects to the catheter. A Foley catheter tends to a have small flow most of the time. While not shown, likewise the left panel 20r can be fully opened along the seam 22r, exposing an interior side of pant 3i, when the right slides 4r,7r are pulled to the stops, see 45r,46r shown in FIG. 1.

The port can be closed by pulling the opposing zipper slides together.

The pulls on the zipper assemblies are locking pulls therein preventing premature closure of the portlet or the port.

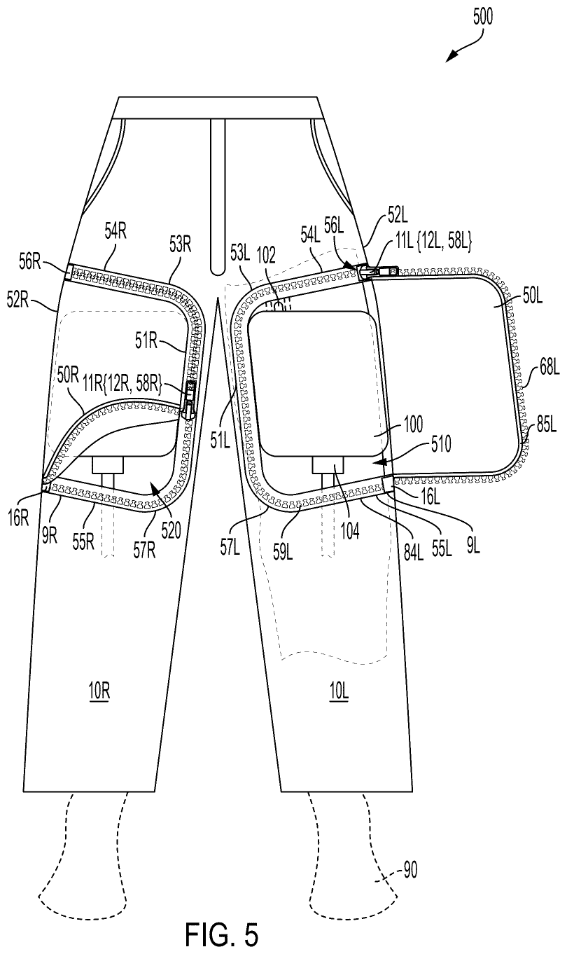

FIG. 5 illustrates pants having a single zipper per pant leg in accordance with embodiments described herein. Similar aspects to the above embodiments are included in this embodiment.

As illustrated in FIG. 5, pants 500 have a left panel 50L and a right panel 50R, therein enabling use and access to two collection bags, or other medical apparatuses that may be attached to a user's leg area. Each panel 50R, 50L is secured by a continuous zipper tape 9R, 9L and a lateral seam 52R, 52L of the pant legs 10R,10L of the pants 500. As described herein, the continuous zipper tape 9R, 9L include a medial section 51R, 51L that upwardly transitions into upper curved section 53R, 53L, which laterally transitions into an upper horizontal sections 54R, 54L, ending in an upper stop 56R,56L. The medial section 51R, 51L also downwardly transitions into a lower curved sections 57R,57L, which laterally transitions into to a lower horizontal section 55R,55L ending in a lower stop 16R,16L. The continuous zipper tapes 9L, 9R span thigh regions of the pants 500, therein connecting three sides of the panels 50L, 50R to corresponding openings of the pants 10R, 10L.

In this configuration, each pant leg 10R, 10L has the continuous zipper tapes 9R, 9L, which taken with the lateral seams 52R, 52L include the panels 50r, 50L. Each of the zipper tapes 9R, 9L has a single zipper slide assembly 11R, 11L; where each zipper slide assembly includes a single zipper slide 12R and 12L with pulls 58R and 58L for opening and closing the panels 50L, 50R in relation to the zipper tapes 9R, 9L. In this alignment when a left slide 12L is pulled from bottom to top, the left panel 50L unzips from the zipper tape 9L and opens, providing a port 510, and when a left slide 12L is pulled from top to bottom, the left panel 50L and zipper tape 9L closes, wherein opposing teeth on the left zipper tape 9L and left panel 50L are alternately interlocked. The zipper assemblies described herein may also be referred to as fastener assemblies.

Likewise, when a right slide 12R is pulled from bottom to top, the right zipper tape 9R and right panel 50R unzips and opens, providing an opening 520 to access the catheter or underlying medical apparatus, and when the right slide 12R is pulled back, the right zipper tape 9R and right panel 50R closes, wherein opposing teeth on the right zipper tape 9R and right panel 50R are alternately interlocked.

The zipper assemblies 11R and 11L are independent, and movement of the right slide 12R does not affect movement of the left slide 12L. This is also the case when the pulls 58R and 58L are locking pulls.

The characteristics about the zipper sizing, configuration, and materials that were described herein for the dual zipper configurations also apply for the single zipper and zipper tape.

The zipper sizes of the dual zippers described herein are also applicable the single zipper design. Similarly, the materials and other configurations of the zippers including locking pulls, zipper tapes, opposing zipper tapes, other types of tapes, coil zippers, and shapes used for the dual zippers may also be used for the single zipper design.

FIG. 5 further illustrates how the left panel 50L may be opened. The zipper slide 12L is pulled, from the left lower stopper position 16L through the left lower horizontal portion 55L, around the lower left curve 57L, through the medial section 51L, around the upper left curve 57L, and then laterally to the upper horizontal section 54L, stopping at a point proximate to the stop 56L, which is proximate to a left seam 52L of the invented pants 500. The unzipped portion of the left panel 50L therein exposes the collection bag 100 and the inlet 102. The unzipped portion of panel 50L in FIG. 5 is bounded by a left set of inner teeth 68L disposed on the left panel 50L and a left set of outer teeth 59L on the zipper tape 9L.

The unzipped portion of the zipper tape 9L provides a panel opening to the inlet 102 and to the collection bag 100. In the illustrated embodiment the outer teeth 59L are on an underlying fabric tape 84L, which provides a protective buffer. Optionally, the inner teeth can also be recessed on an overlying fabric tape 85L.

The unzipped panel 50L is folded laterally along the lateral seam of the pant leg, providing the port 510 with full access to the collection bag. The left panel 50L may be fully opened along the seam 52L, exposing an inside of pant 10L, when the left slide 12L is pulled from the lower stop 16L to the upper stop 56L. With the left panel 50L fully opened one can view the collection bag 100, the inlet 102 and the outlet 104. A view of how much urine has been collected is possible, depending on how much of the port 510 is exposed.

The port 510 can be closed by pulling the slide 12L down from the top to the bottom. The pulls 58L on the zipper assemblies 11L may be locking pulls configured to prevent premature closure of the port 510.

While FIG. 5 illustrates two panel regions to be accessed by a user to access underlying medical appratuses, embodiments are not limited thereto. However, embodiments described herein may also be used with only a single pant leg having an access panel disposed on either the left pant leg 10L or right pant leg 10R.

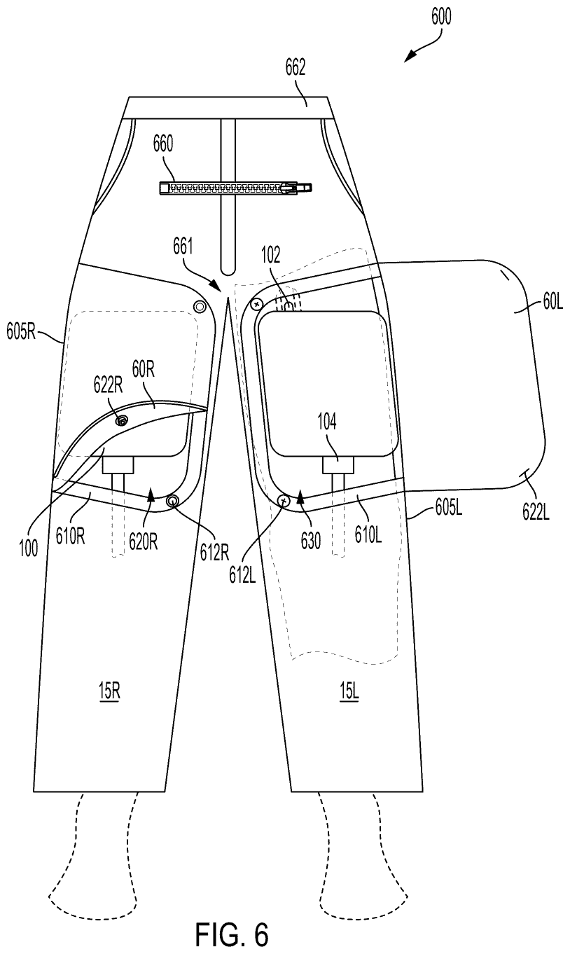

FIG. 6 illustrates another embodiment of pants 600 described herein. Similar aspects to the above embodiments are included in this embodiment. The illustrated pants 600 of FIG. 6 have a left panel 60L and a right panel 60R attached to respective seams 605L and 605R of pant legs 15R and 15L, therein enabling use and access to one or two collection bags disposed therein.

The right panel 60R is configured to be secured to the pants 600 by the right seam 605R on one edge and secured by a continuous snap tape 610R on the body of the pant leg 15R. The snap tape 610R includes a plurality of snap sockets 612R. The snap sockets 612R are a “female” part of a snap configuration and may be known by other terms as is known in the art. The right panel 60R includes a plurality of snap caps 622R configured to snap into corresponding snap sockets 612R on the snap tape 610R. The snap caps 622R are a “male” part of the snap configuration and may be known by other terms as is known in the art. In operation, a user may open a portlet 620R or larger size hole to inspect a volume content of a urine bag 100, as discussed herein. Though the left pant leg 15L is illustrated with another embodiment having a button configuration, either pant leg may be configured with the snaps or buttons.

The left panel 60L is configured to be secured to the pants 600 by the left seam 605L on one edge and secured by a continuous button tape 610L on the body of pant leg 15L. The button tape 610L includes a plurality of buttons 612L sewn or otherwise mounted to the button tape 610L. The left panel 60L includes a plurality of button slits 622L configured to receive the plurality of buttons 612L and fasten the left panel 60L to the body of the pant leg 15L. In operation, a user may open a portlet or larger size hole 630 to inspect a urine bag or the contents therein.

The continuous snap tape 610R and the continuous button tape 610L may also each be known as a fastener tape. The fastener tape represents that both the snap mechanism and button mechanisms are fastener assemblies. The respective combinations of button slits 622L, buttons 612L, snap caps 622R, and snap sockets 612R may be referred to as engagement mechanisms.

Because the snap tape 610R and the button tape 610L are configured in a similar manner to the continuous zipper tapes discussed herein, the corresponding sections of the tapes 610R, 610L may also be referred to as medial section, upper curved section, upper horizontal section, lower curved section, and lower horizontal section.

As noted with the zipper designs, the continuous snap tape 610R and button tape 610L are continuous with an outer edge of the respective panels 60R, 60L with an adjoining inner edge of an opening of the pant, therein connecting to the edge of the panel to the inner edge of the opening of the pant.

The snap male and female parts as well as the button and slit sizing may be varied to suit the desires of a customer or manufacturer. Illustrated are two snap mechanisms and two buttons per pant leg, but embodiments are not limited thereto. The snap tape 610R and button tape 610L may be configured with up to a dozen snaps or buttons with which to fasten the respective panels 60R or 60L to the tapes.

Also, the male and female connectors 612R and 622R of the snap tape 610R and panel 60R may be reversed such that the male part 622R is disposed on the snap tape 610R and the female part 612R is disposed on the panel 60R. Similarly for the left panel 60L, the buttons 612L may be disposed on the panel 60L and the slits 622L may be disposed on the button tape 610L.

As illustrated in FIG. 6, the partially snapped panel 60R is folded laterally along the lateral seam 605R of the pant leg 15R, providing a portlet 620R with partial access to the collection bag via the portlet 620R.

On the other pant leg 15L, the left panel 60L is fully opened along the seam 605L, exposing an inside of the pant leg 65L. With the left panel 60L fully opened one can view the collection bag 100, the inlet 102 and the outlet 104. A view of how much urine has been collected, a partial view of the straps is possible, and an inspection of the connection of the inlet to a catheter. The port 630 and portlet 620 may be closed by snapping or buttoning the opposing sides together.

Another feature of embodiments described herein is an access slot 660 disposed in the pants 600. Although illustrated in pants 600, any of the embodiments described herein may include the access slot 660.

The access slot 660 is an opening in an upper region of the pants 600. The access slot 660 is positioned between an intersection 661 where the right pant leg 15 and left pant leg 15L meet, and a waistline 662 of the pants 600. The access slot 660 may be positioned about three inches below the waistline 662. A length of the access slot 660 may be on the order of eight inches long to allow a user to open a wide area of the pants 600 to access a dressing underneath, adjust a catheter tube, clean a catheter tube, or the like. The access slot 660 may be configured with different fastener assemblies described herein such as a zipper slider and locking pull, a snap assembly, a button assembly, or with hook and loop portions to access an underlying area.

The access slot 660 provides to a wearer of the pants 600, a helper, or health care provider to be able to access a location of a medical dressing on a patient. The person accessing the dressing location may change the dressing or may access a tube of a Foley Catheter for a patient, for such reasons as maintenance or cleaning, or for flushing the catheter. The access slot 660 represents a general position where a catheter may enter the bladder of a patient and is designed to allow a user to access this area to treat a patient.

Though FIG. 6 illustrates the access slot 660, as noted herein, the access slot 660 is an option for the designer of the pants to be used. A customer or manufacturer may customize different aspects of the pants described herein with different variations of fastener assemblies and access slots.

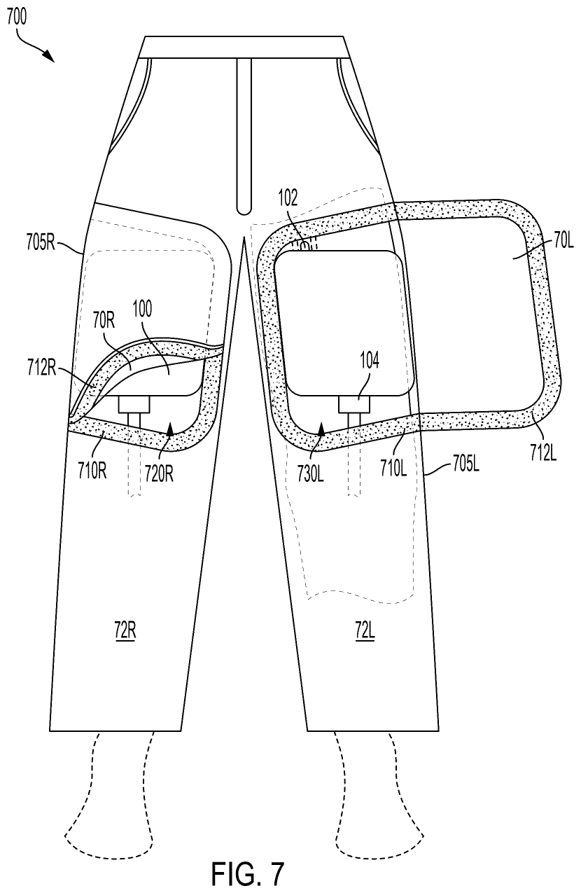

FIG. 7 illustrates another embodiment of pants 700 described herein. The illustrated pants 700 of FIG. 7 have a left panel 70L and a right panel 70R attached to respective pant seams 705L and 705R, therein enabling use and access to one or two collection bags disposed therein.

The right panel 70R is configured to be secured to the pants 700 by the right seam 705R on one edge and secured by a continuous hook and loop tape 710R on the body of pant leg 70R, such as a Velcro® tape. The tape 710R can be the hook side, the rough, scratchy side with tiny hooks that latch onto the loops on the other side. The hook and loop tape 710R can be the loops side, the soft, fuzzy side made of tiny loops that catch the hooks from the hook side. The right panel 70R includes an opposing hook and loop part 712R to correspond to the hook and loop tape 710R. In operation, a user may open a portlet 720R or larger size hole to inspect a volume content of the urine bag 100, as discussed herein. The hook and loop tapes described herein are also one example of fastener assemblies.

The left panel 70L is also configured to be secured to the pants 700 by the left seam 705L on one edge and secured by a continuous hook and loop tape 710L on the body of pant leg 70L. The left panel 70L includes the opposing part of the hook and loop tape along an edge thereof, to correspond to the hook and loop on the tape 710L.

The hook and loop tapes 710L, 712L and the opposing sides may also be referred to a fastening members as discussed herein. Each side of the hook and loop tapes 710L, 712L and opposing sides may be considered a plurality of fastening members because in a hook and loop tape there are a plurality of hooks and opposing loops that make them up.

In operation, a user may open a portlet or larger size hole 730 to inspect a urine bag or the contents therein.

The width of the hook and loop tapes may be varied to suit the desires of a customer or manufacturer, but in general may be about one inch width.

As illustrated in FIG. 7, the partially opened panel 70R may be folded laterally along the lateral seam 705R of the pant leg 72R, providing the user partial access to the collection bag 100 via the portlet 720R. The left panel 70L is illustrated as fully opened connected to the seam 705L, exposing an inside of the pant 72L.

With the left panel 70L fully opened one can view the collection bag 100, the inlet 102 and the outlet 104. A view of how much urine has been collected, a partial view of the straps is possible, and an inspection of the connection of the inlet to a catheter.

The port 730R and portlet 720R can be closed by fastening the two sides together.

Although the present disclosure has been illustrated and described herein with reference to exemplary embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims.

Finally, any numerical parameters set forth in this Specification and the attached Claims are approximations (for example, by using the term “about”) that may vary depending upon the desired properties sought to be obtained by the present invention. At the very least, and not as an attempt to limit the application of the doctrine of equivalents to the scope of the Claims, each numerical parameter should be construed in light of the number of significant digits and by applying ordinary rounding.

It is to be understood that the foregoing description and specific embodiments are merely illustrative of the best mode of the invention and the principles thereof, and that various modifications and additions may be made to the invention by those skilled in the art, without departing from the spirit and scope of this invention, which is therefore understood to be limited only by the scope of the appended claims.

1. Pants that can accommodate a catheter, wherein the catheter is configured to drain urine from a wearer's bladder to at least one collection bag, said pants comprising:
a panel that is a covering component of a pant leg that can provide access to the at least one collection bag on a frontal anterior thigh of the wearer, wherein said panel provides partial and full access to the at least one collection bag;
wherein said panel has a lateral edge that is coextensive with a lateral seam of a pant, a partial perimeter outer edge that is closed by a continuous zipper tape with a stop, where the partial perimeter outer edge of said panel is substantially coextensive with an inner edge of the pant leg;
said continuous zipper tape has a zipper slide assembly with a zipper slide having a locking pull configured to open and close the panel and continuous zipper tape;
wherein the zipper slide is pulled to provide a port at a position along the continuous zipper tape and the panel is folded laterally along the lateral seam of the pant leg.

2. The pants according to claim 1, further comprising:
an abdominal region, which is large enough to accommodate the catheter.

3. The pants according to claim 1, further comprising a fabric that is sufficiently supple that the wearer can feel the catheter through the fabric, to determine if the catheter needs adjustment.

4. The pants according to claim 1, wherein the continuous zipper tape comprises:
a medial section that upwardly transitions into an upper curved section, which laterally transitions into an upper horizontal section ending in the upper stop; and
the medial section also downwardly transitions into a lower curved section, which laterally transitions into to a lower horizontal section ending in the lower stop.

5. The pants according to claim 1, further comprising:
a left panel on a left pant that is substantially a mirror image of a right panel on a right pant.

6. The pants according to claim 1, wherein the continuous zipper tape comprises a coil zipper tape.

7. The pants according to claim 1, wherein the pulls on the zipper assemblies comprise locking pulls configured to prevent accidental closure of the portlet or the port.

8. The pants according to claim 1, further comprising:
a length of flexible plastic tubing that is several inches long, therein extending the outlet of the collection bag below a wearer's knee, thereby facilitating periodic drainage into a commode, urinal or a bedpan, or any suitable vessel.

9. The pants according to claim 6, further wherein said continuous zipper tape is selected from the group consisting of: a size #4, #4.5 or #5 for most pants.

10. The pants according to claim 6, further comprising:
said continuous zipper tape is selected from size #10±#3 for very large pants.

11. The pants according to claim 1, wherein the continuous zipper tape comprises:
a combination of outer teeth mounted on a panel's side tape and inner teeth mounted on a pant's side tape.

12. Pants that can accommodate a catheter, wherein the catheter is configured to drain urine from a wearer's bladder to at least one collection bag, said pants comprising:
a panel that is a covering component of a pant leg that can provide access to the at least one collection bag on a frontal anterior thigh of the wearer, wherein said panel provides partial and full access to the at least one collection bag as needed;
wherein said panel has a lateral edge that is coextensive with a lateral seam of a pant, a partial perimeter outer edge that is closed by a continuous fastener tape with a stop, where the partial perimeter outer edge of said panel is substantially coextensive with an inner edge of the pant leg;
said continuous fastener tape has a fastener assembly with a plurality of engagement mechanisms for opening and closing said continuous fastener tape, and by extension said panel;
a first operating element of the fastener assembly is that as at least one engagement mechanism is pulled apart, the continuous fastener tape separates, therein providing a portlet at a position along the said continuous fastener tape;
wherein the portlet can be closed by fastening the at least one engagement mechanisms together; and
a second operating element of the fastener assembly is that when all of the engagement mechanisms are pulled apart, then the fastener tape is unengaged therein providing a port, and the panel can be folded laterally along the lateral seam of the pant leg; and
wherein the port can be closed by engaging the plurality of engagement mechanisms together.

13. The pants according to claim 12, wherein the fastener tape is a snap tape and the engagement mechanism is a snap cap and a snap socket.

14. The pants according to claim 12, wherein the fastener tape is a button tape and the engagement mechanism is a button and a slit.

15. The pants according to claim 12, wherein the fastener tape and an opposing side thereof on the panel are a hook and loop tape.
